(19) United States
(12) Patent Application Publication
(10) Pub. No.: US 2025/0067715 A1
(43) Pub. Date: Feb. 27, 2025

(54) MONITORING SYSTEM WITH A GAS COLLECTION DEVICE

(71) Applicant: Dräger Safety AG & Co. KGaA, Lübeck (DE)

(72) Inventors: Tilman von BLUMENTHAL, Lübeck (DE); Hans-Ullrich HANSMANN, Lübeck (DE); Henning GERDER, Lübeck (DE); Robert JAHNS, Lübeck (DE); Christoph OSTERLOH, Lübeck (DE)

(21) Appl. No.: 18/811,945

(22) Filed: Aug. 22, 2024

(30) Foreign Application Priority Data
Aug. 23, 2023 (DE) 10 2023 122 581.2

(51) Int. Cl. G01N 33/00; G01N 1/22

(52) U.S. Cl. CPC G01N 33/0062; G01N 1/2247

(57) ABSTRACT

A monitoring system (100) has an associated gas collection device (81) for use in a breathing gas supply in aircraft. A process (900) provides coordinated operation of the monitoring system (100) and gas collection device (81). During coordinated operation of the monitoring system (100) and gas collection device (81), a module (50) for gas transportation is activated (902) and the module (55) for gas quantity control is activated (903) for supplying defined quantities (80) of breathing gas mixture to the gas collection device (81) with the gas collection containers (82, 83, 84).

### CROSS REFERENCE TO RELATED APPLICATIONS

This application claims the benefit of priority under 35 U.S.C. § 119 of German Application 10 2023 122 581.2, filed Aug. 23, 2023, the entire contents of which are incorporated herein by reference.

### TECHNICAL FIELD

The present invention relates to a monitoring system with a gas collection device and a process for operating a monitoring system. The monitoring system is used to monitor the breathing gas supply of an aircraft pilot in an aircraft. Aircraft or flying devices (aircraft) are to be understood as airplanes or helicopters in civil or military aviation, such as passenger airplanes in scheduled or charter traffic as well as ultra-fast airplanes close to or above the range of supersonic speed. In particular, flights with jet aircraft (jets) at supersonic speeds and/or at altitudes above 15,000 meters above sea level, place great demands on the fitness to fly of flight personnel. Fitness to fly with physical and mental fitness, alertness, concentration and vigilance must be ensured at all times at high altitudes, rapid flight maneuvers or flight attitudes, such as turns, dives, inverted flight at high speeds and high accelerations. In addition to the personal and health conditions of the pilot and reliable aircraft equipment, a reliable supply of clean and safe breathing air for the pilot is also essential to ensure safe flight operations.

### BACKGROUND

Paramagnetic processes are often used to determine the oxygen concentration in gases. A basic principle for measuring oxygen in a measuring chamber using heat conduction changes in connection with paramagnetism is described in U.S. Pat. No. 6,430,987B1. US20230114548A1 describes a way of determining the presence of an anesthetic gas in the breathing gas mixture in addition to measuring oxygen.

Paramagnetic measuring devices for determining oxygen concentrations, in particular also in breathing gases, are known, for example, from U.S. Pat. No. 6,952,947 B2, US2011094293 A1, U.S. Pat. Nos. 8,596,109 B2, 9,360,441 B2, 6,895,802 B2, 6,405,578 B2, 6,430,987 B1, 4,808,921 A, 4,683,426 A, 3,646,803 A, 3,584,499 A, 2,944,418 A.

It is also known to measure the composition of respiratory gases optically.

The absorption of light in a certain wavelength range specific to the respective gas serves as a measure of the concentration of the respective gas. U.S. Pat. No. 5,739,535 A describes an infrared optical gas measuring device. An infrared-optical carbon dioxide sensor, a so-called IR carbon dioxide sensor, is known from U.S. Pat. No. 8,399,839 B2. U.S. Pat. No. 6,571,622 B2 describes a combination sensor comprising an infrared-optical carbon dioxide sensor with a flow sensor, which can be arranged in the main stream in the respiratory gas path of a patient.

U.S. Pat. Nos. 2,004,238 746 A1, 2,002,036 266 A1 are known infrared-optical carbon dioxide sensors which can be arranged in the side stream in or on the respiratory gas path of a patient.

U.S. Pat. Nos. 6,954,702 B2, 7,606,668 B2, 8,080,798 B2, 7,501,630 B2, 7,684,931 B2, 7,432,508 B2, 7,183,552 B2 show gas measuring systems for detecting gas concentrations in the side stream and main stream.

EP 0 042 683 B1 discloses a process for producing reusable, adsorptive test tubes for gases and vapors. The size of the test tubes is such that they can be worn on the body for personal use.

EP 2 739 954 B1 discloses a device for measuring gases escaping from underground sources.

U.S. Pat. No. 4,040,783 A discloses a device for analyzing the emission content of the exhaust gases of an internal combustion engine, wherein a certain volume portion of the exhaust gases is separated and collected in an intermediate container. A portion of the volume is passed through gas analyzer tubes containing a medium suitable for indicating a particular contaminant in the gas.

US 2020/141876 A1 discloses a kit for calorimetric gas detector tubes or test tubes. Gas detector tubes can be used to either visually indicate the concentration of target gases and/or interfering gases in a gas sample. Gas detector devices usually consist of a transparent tube containing a chemical reagent that shows a color change on contact with a target gas. After adding a sample of the target gas from the environment, the resulting color change can be read.

U.S. Pat. No. 5,397,538 A shows a device for the colorimetric detection of gaseous and/or vaporous components of a gas mixture from the coloration of a channel-shaped reaction zone, one or more of which are accommodated on a plate-shaped reagent carrier.

Monitoring systems for monitoring pilots and/or co-pilots are known from US 2021/0405008 A1 and US 2023/285779 A1. The monitoring systems are preferably designed as part of the pilot's equipment as a self-sufficient and mobile unit worn on the body with an independent power supply.

Oxygen supply systems for aviation have been available and known in the state of the art for some time. Quantities of oxygen for supplying the pilot can be kept available in gaseous (GOX system) or liquid form (LOX system). Alternative systems continuously generate the required quantities of oxygen themselves during the flight. Such devices are known as OBOGS (On-Board Oxygen Generation Systems).

US 2004/245390 A1 shows an emergency oxygen supply system for an aircraft. In addition to an on-board breathing gas supply in the form of a pressurized gas source, oxygen can be provided by a molecular bed sieve arrangement.

U.S. Pat. No. 10,561,863 B1 discloses a wearable device for monitoring physiological metrics to determine metabolic, pulmonary and cardiac function and to measure oxygen saturation. The device monitors a physiological profile of a person and is capable of detecting physiological changes, predicting the onset of symptoms, and alerting the wearer or another person or system.

A variant of a monitoring system for monitoring a pilot and/or co-pilot is known from the German patent application DE 10 2023 121 409.8, which is designed to determine whether there is a presence of foreign gas components in a breathing gas mixture.

Foreign gas components in the breathing gas mixture can, for example, be present in the form of various fluids, gases or gas mixtures. Examples include methane, butane, propane, ethane, butane-propane mixtures, operating fluids and fuels such as kerosene, petrol, natural gas, liquid gas, operating fluids such as coolants and lubricants, as well as hydraulic fluids, lubricants or anti-corrosion agents. The fuels or coolants and lubricants can be based on compositions of hydrocarbons (C Hₘₙ), halogenated hydrocarbons and chlorofluorocarbons. Hydraulic fluids, lubricants or lubricants can be based, for example, on polymerized fluorocarbons, such as fluoropolymers (e.g. polytetrafluoroethylene PTFE).

During operation of the aircraft or aircraft equipment (flying devices), the operating fluids or operating materials can potentially enter the breathing gas supply of the aircraft or aircraft equipment directly or their reaction products or vapors. The potential constituents could, for example, be components from the exhaust gas of the combustion process. For example, exhaust gases and reaction products from the turbines of a jet aircraft flying ahead could potentially enter the intake tract of the breathing gas supply of an aircraft flying behind. Components in the exhaust gas are, for example, unburned kerosene, mainly carbon dioxide and also water vapor. Comparable situations in which unburned fuel can enter the breathing gas supply can arise during refueling in the air. Other substances that may enter the breathing gas supply during flight operations can come from the compressor stages of the turbines, engines or other units, for example. During operation, various coolants and lubricants, often based on hydrocarbons, as well as hydraulic fluids are usually used as fluids. In addition, various different isomers of tricresyl phosphate (TCP) can be formed as a reaction product of the coolants, lubricants or hydraulic fluids, for example through the effect of heat. TCP can pose a health risk in the event of inhalation, skin or eye contact, and the effects of other chemical substances or groups of substances that can be produced as derivatives or reaction products during engine operation cannot necessarily be considered harmless.

In particular, the monitoring system can detect the presence of another gas other than carbon dioxide, nitrogen, moisture or water vapor and oxygen and provide an output signal indicating this particular condition. Further information is required to assess whether, in the event that a certain special condition of this type is present with the presence of a further gas in the breathing gas mixture, which is different from carbon dioxide, nitrogen, moisture or water vapor and oxygen, and whether the breathing gas mixture meets the requirements for health safety for the pilot or co-pilot. This further information should also include the nature or type of the foreign gas components in the breathing gas mixture.

### SUMMARY

Based on the state of the art, it is an object of the invention to identify the presence of foreign gas components in the breathing gas supply of an aircraft pilot.

This object is attained by a monitoring system with a gas collection device with features according to the invention, as well as by a process with features according to the invention.

Further features and details of the invention can be seen from this disclosure, including the description, the claims and the drawings. Features and details which are described in connection with the monitoring system according to the invention naturally also apply in connection with the process according to the invention and vice versa, so that reference is or can always be made reciprocally to the individual aspects of the invention with regard to the disclosure.

A monitoring system according to the invention for monitoring the composition of a breathing gas mixture of a breathing gas supply of an aircraft pilot in aircraft or flying devices has a sensor system, a gas transportation module, a gas quantity control module and a control and evaluation unit.

According to a first aspect of the invention, embodiments are disclosed, which include a monitoring system which has at least one sensor system, a gas transportation module, a gas quantity control module and a control and evaluation unit, as well as a gas collection device assigned to the monitoring system.

The sensor system has at least one gas sensor. The at least one gas sensor is configured to determine proportions of oxygen (O₂), moisture or water vapor (H₂O), nitrogen (N₂) or carbon dioxide (CO₂) in the breathing gas mixture. The sensor system can also have a humidity sensor (moisture sensor) for determining a humidity in the breathing gas mixture, which in combination with the control and evaluation unit is configured to determine a value that indicates a humidity or a proportion of water in the breathing gas mixture.

The sensor system can also have a pressure sensor for determining a pressure level in the breathing gas mixture, which in combination with the control and evaluation unit is configured to determine a value that indicates the pressure level of the breathing gas mixture. The sensor system can also have a temperature sensor for determining a temperature of the breathing gas mixture, which, in combination with the control and evaluation unit, is configured to determine a value that indicates a temperature of the breathing gas mixture.

The control and evaluation unit is configured to organize, monitor, control or regulate the monitoring of the breathing gas supply. The control and evaluation unit is also configured to control the gas transportation module and the gas quantity control module.

The module is configured to supply defined quantities of breathing gas mixture from a measuring location (measuring point) to the monitoring system and to the sensor system by means of a sample gas line (measuring gas line). The gas transportation module is also configured to supply quantities of breathing gas mixture to the gas quantity control module. The gas quantity control module is connected to the gas collection device by means of a collection gas line. The gas quantity control module is configured to control the supply of defined quantities of breathing gas mixture from the monitoring system to the gas collection device. The gas quantity control module is configured to provide the defined quantities of breathing gas mixture by means of a gas outlet of a collection gas line arranged on the monitoring system and a gas inlet of the gas collection device arranged on the gas collection device. The gas collection device has at least one collection container in the form of an adsorption tube, into which the defined quantities of breathing gas mixture flow and can then be collected and stored. A gas distribution system can be arranged in the gas collection device, which enables distribution of the defined quantities of breathing gas mixture to a plurality of collection containers arranged in the gas collection device.

The at least one gas collection device is configured to collect and store quantities of the breathing gas mixture. For this purpose, the at least one gas collection device can have at least one collection container. Such a collection container can preferably be configured as an adsorption tube for gas sampling.

Adsorption tubes are configured to collect quantities of a gas sample or a sample of a breathing gas mixture and to store these quantities for gas analysis at a significantly later point in time and then make them available for gas analysis, for example by a gas chromatograph. Collection containers can be configured as silica gel collection tubes, for example. An adsorption tube with polymer resins as adsorbents, for example in the form of poly(2,6-diphenyl-p-phenylene oxide), is also particularly suitable for collection and/or pre-concentrating volatile organic compounds (VOCs). Poly(2,6-diphenyl-p-phenylene oxide) is also known under the brand name “Tenax”.

Embodiments show how the interaction, in particular a coordinated interaction, between the gas collection device and the monitoring system can be configured.

In a preferred embodiment, the control and evaluation unit can be configured as a plurality of control modules. At least one further control module can be configured as a component of the at least one gas collection device.

In preferred embodiments, at least one interface can be arranged in or on the monitoring system and/or the gas collection device. The interface is preferably configured as a power and/or data interface.

The control and evaluation unit can be configured to coordinate the gas collection device and monitoring system by means of the interfaces. In this preferred embodiment, components of the monitoring system and gas collection device can be connected to each other by means of interfaces for energy and/or data transfer. The data transfer can be unidirectional from the monitoring system to the gas collection device, as well as bidirectional between the monitoring system and the gas collection device.

By means of a unidirectional data connection from the monitoring system to the gas collection device, for example, the monitoring system can control or trigger the gas collection device to activate the collection of quantities of breathing gas mixture into the adsorption tubes.

In a preferred embodiment, the control and evaluation unit and/or the control modules can be configured to receive a status signal at the at least one interface and to take the status signal into account when performing the coordination. In such preferred embodiments, the control and evaluation unit can perform activation and/or deactivation of a switching device depending on measured values determined with the monitoring system and/or states of the breathing gas supply identified with the monitoring system.

In a preferred embodiment, the control and evaluation unit can be configured to execute at least one action from the following group of actions when performing the coordination:
Activation of a switching device (switching means) or a changeover device (changeover means) to start the supply of quantities of breathing gas mixture to the gas collection device;
Deactivation of the switching device or changeover device to terminate the supply of quantities of breathing gas mixture to the gas collection device;
Activation of the gas transportation module;
Activation of the gas quantity control module and/or other components of the gas collection device for supplying and collecting quantities of breathing gases in the gas collection device.

In a preferred embodiment, the at least one collection container can comprise an arrangement with a plurality of collection containers. At least one collection container or a plurality of collection containers can be arranged in the gas collection device. The collection containers can, for example, be configured as absorption tubes, often also called collection tubes. Several collection tubes can be arranged in a parallel or serial arrangement in the gas collection device.

In special embodiments, different types of gas collection tubes can also be used, for example to detect different substances in the breathing gas mixture. For example, gas collection tubes for various foreign substances in the breathing gas mixture, that may be expected during flight operations, can be used to obtain the best possible picture of the gas composition in the breathing gas mixture following flight operations with the aid of the collection tubes in the laboratory.

In a preferred embodiment, the further control module of the gas collection device can be configured with a switching device arranged in the gas collection device for distributing the quantities of breathing gas mixture to the collection containers of the gas collection device. The switching device can ensure that the supplied quantities of breathing gas mixture are distributed to individual collection containers (collection tubes) of the large number of collection containers. Valve arrangements, such as 2/3-way valves or changeover valves, are possible switching devices.

In a preferred embodiment, the control and evaluation unit and/or the control modules can coordinate the activation and/or deactivation of the switching device as a function of measured values determined by the monitoring system and/or states of the breathing gas supply identified by the monitoring system. If, for example, a situation or condition has been determined or identified by the monitoring system that foreign gas components are present in the breathing gas mixture, the monitoring system can initiate a supply of quantities of breathing gas mixture to the gas collection device and activate the switching device in the gas collection device with the aid of control lines in such a way that quantities of breathing gas mixture are distributed and/or supplied to the collection containers. In this way, it can be ensured that quantities of breathing gas mixture can only flow into the collection tubes and be collected there if an event has occurred that justifies the collection of breathing gas mixture. This means that the capacity of collection tubes can be kept as low as necessary. This has the particular advantage that the amount of equipment for the pilot during flight operations can also be reduced to the absolutely necessary equipment, i.e. with the gas collection device in combination with the monitoring system for analyzing the breathing gas mixture.

In a particularly preferred embodiment, an additional sensor system can be arranged in the monitoring system, which is configured to determine events and/or situations of the flight operation. The control and evaluation unit or one of the control modules coordinates the activation and/or deactivation of the switching device depending on the determined events and/or situations of the flight operation. In this way, it is possible to activate the collection of quantities of breathing gas mixture in the collection containers for selected events or situations during flight operations.

The additional sensor system can, for example, have an acceleration sensor, such as a 2- or 3-axis acceleration sensor (accelerometer), a compass sensor, such as an electronic compass, gyrocompass or fluxgate compass, an altitude sensor (altimeter) or a gyro sensor (gyrometer). The other sensors can be used to identify flight maneuvers, flight situations (take-off, landing, descent, acceleration, looping, refueling in the air) and serve as triggers for initiating the activation and deactivation of the switching device that are responsible for supplying and/or collecting quantities of breathing gas mixture in the collection containers.

In a preferred embodiment, an output unit can be arranged in or on the monitoring system or assigned to the monitoring system, which enables the provision of an output signal that indicates an operating state of the gas collection device. Indicated states of the gas collection device—in addition to states that indicate operational readiness or malfunctions—can be, for example, the current actuations, current uses or utilization, as well as filling situations (empty, 50% full, 100% full) of the collection containers, i.e. in particular in the form of the plurality of collection containers, collection tubes or adsorption tubes.

The described embodiments with monitoring system and gas collection device therefore demonstrate the advantages particularly well when the monitoring system, gas collection device and optional additional sensors are coordinated.

Embodiments relating to the monitoring system and/or the gas collection device have been described and explained above.

A process for operating the monitoring system and the gas collection device is described below as a further inventive aspect.

The process according to the invention can, for example, be carried out by a control and evaluation unit or control modules, as described for the monitoring system according to the invention and/or the gas collection device as well as the embodiments of the monitoring system and/or the gas collection device.

The same, similar, the same or additional advantages as described in the embodiments of a monitoring system with associated gas collection device can be obtained by the process according to the invention and its embodiments.

In this respect, please refer to the information provided there with regard to the benefits achieved.

In carrying out the process for operating a monitoring system with a gas collection device for monitoring a breathing gas supply of an aircraft pilot in aircraft or flying devices, the following steps are carried out in a step sequence:
a) Activation of the switching device or the changeover device to start the supply of quantities of breathing gas to the gas collection device;
b) Deactivation of the switching device or the changeover device to terminate the supply of quantities of breathing gas to the gas collection device;
c) Activation of components of the gas collection device to collect quantities of breathing gases;
d) Provision of an output signal which indicates an operating state of the gas collection device.

The start of the process with the sequence of steps a)-d) can be initiated as a function of a status signal provided, which indicates a status of the breathing gas supply of a pilot in aircraft or flying devices or indicates events or situations of flight operations.

In a preferred embodiment of the process, in a further step, quantities of breathing gas mixture can be distributed and/or divided into different gas collection containers.

In a preferred embodiment of the process, an output signal can be provided in a further step, which indicates an operating state of the gas collection device.

The invention is explained in more detail in the following description with partial reference to the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a mask arrangement with a gas port, a connection element as well as hose lines and a gas sample line according to the state of the art;

FIG. 2 is a schematic view showing a monitoring system with a gas collection device; and

FIG. 3 is a flow diagram showing a coordination sequence with a gas collection device.

### DESCRIPTION OF PREFERRED EMBODIMENTS

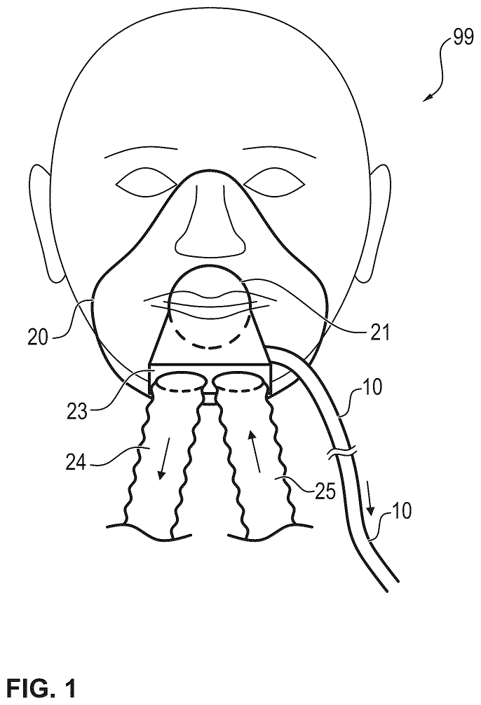

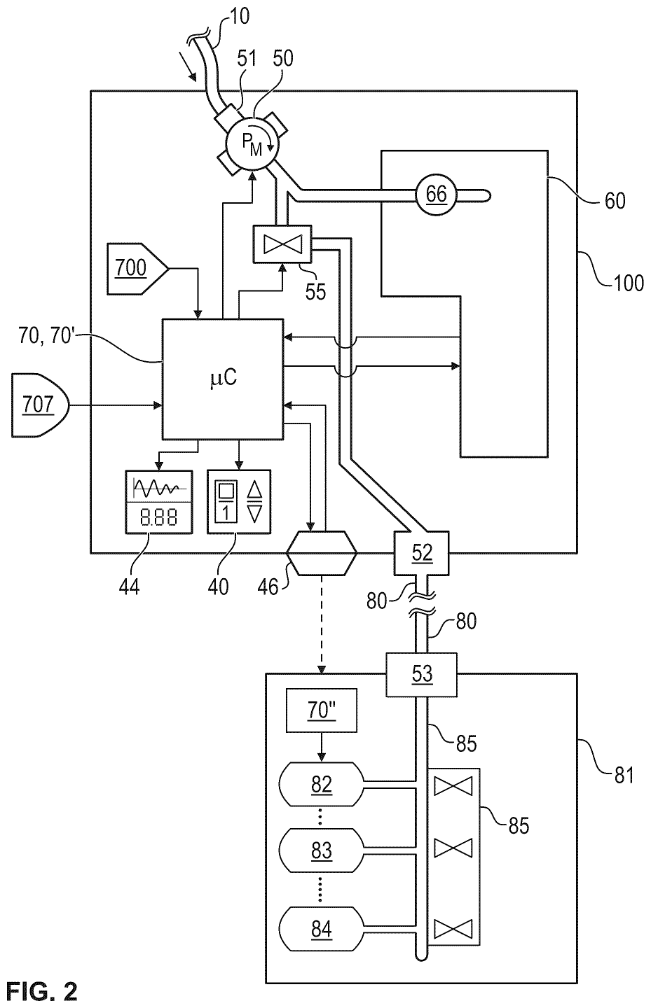

Referring to the drawings, FIG. 1 shows a schematic representation of a breathing gas arrangement. FIG. 2 shows a monitoring system 100 according to the invention. The monitoring system 100 is connected to a person 99 with a sample gas line 10 by means of a breathing mask 20.

The person 99 represents an aircraft pilot (pilot, co-pilot). The breathing mask arrangement includes the breathing mask 20 that has a gas connection 21, a connection element 23 and hose lines (tubes) 24, 25. The hose lines 24, 25 are used to remove and supply breathing gases to the person 99.

The monitoring system 100 has operating and input elements 40, display elements 44, at least one gas transportation module 50, a sensor system 60 with at least one gas sensor 66. The module for the gas inlet 50 is preferably configured as a pump P_{M}.

In addition, the monitoring system 100 has a control and evaluation unit 70.

The operating and input elements 40, the display elements (indicators) 44, the sensors 60 and the gas transportation module 50 are connected to the control and evaluation unit 70 via signal and data lines or control lines.

These control lines or signal and data lines can be configured as a bus system (CAN) or network, for example.

The control and evaluation unit 70 is configured and intended to control and/or actuate the gas transportation module 50 in such a way that breathing gases are conveyed from the breathing mask 20 through the sample gas line 10 and a gas inlet 51 to the sensor system 60.

Thus, the at least one gas sensor 66 in the sensor system 60 then has a quantity or partial quantity of breathing gas available in order to detect and/or analyze the breathing gas metrologically and provide the analysis to the control and evaluation unit 70 as measured values. By means of the control and evaluation unit 70, it is possible to evaluate and process the measured values and display them on display elements 44.

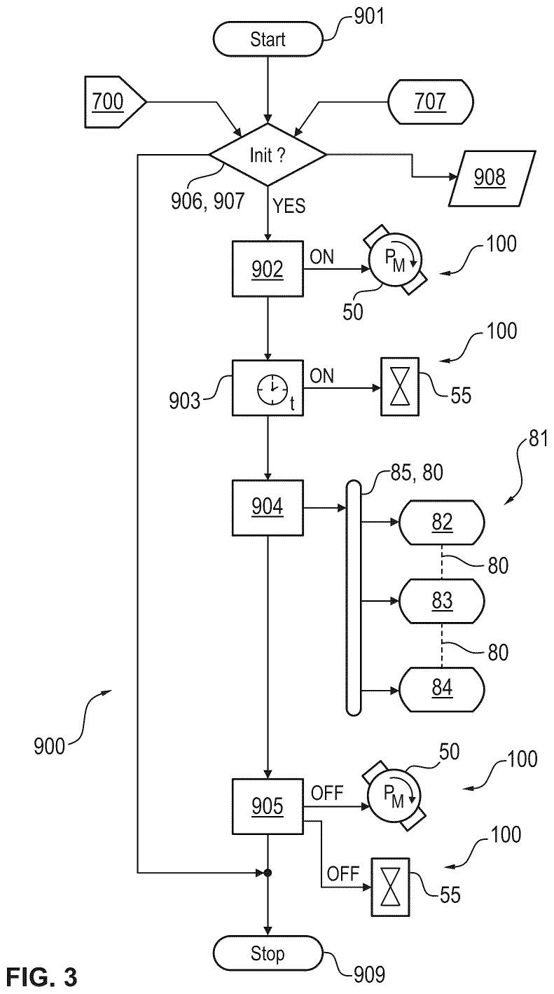

The operating and input elements 40 make it possible to configure the operation 900 (FIG. 3) of the monitoring system 100 with the gas collection device 81 (FIG. 2).

FIG. 2 shows a monitoring system 100 in combination with a gas collection device 81. The gas collection device 81 has collection containers or collection vessels 82, 83, 84 (collection tubes, absorption tubes).

Quantities of breathing gas mixture are supplied via a gas outlet 52 and a collection gas line 80 from the monitoring system 100 via an inlet 53 in the gas collection device 81 to a gas distribution system 85.

The gas distribution system 85 is used for distribution of the breathing gas mixture to the collection containers 82, 83, 84. A control module 70″ is shown in the gas collection device 81, which coordinates the gas distribution system 85 to control the distribution of the quantities of breathing gas mixture from the collection gas line 80 into the collection containers 82, 83, 84 in practice as an example.

The control module 70′, 70″ can be configured as a submodule of the control and evaluation unit 70, for example in a master/slave arrangement, whereby the control and evaluation unit 70 preferably forms the master function, whereby the coordination 900 (FIG. 3) of the cooperation between the monitoring system 100 and the gas collection device 81 is thus essentially the responsibility of the monitoring system 100.

In particular embodiments, the control and evaluation unit 70 can also have a control module 70′ as a submodule in the monitoring system 100 itself.

The control and evaluation unit 70 is configured to carry out the coordination 903, 905 (FIG. 3) and the cooperation between the monitoring system 100 and the gas collection device 81 via an interface 46. The control and evaluation unit 70 is configured and intended to control and/or actuate the module 50 for gas transportation in such a way that gas delivery, i.e. transportation of quantities or molecules of breathing gas through the sample gas line 10 and the gas inlet 51 to the sensor system 60, is achieved.

The control and evaluation unit 70 is also configured to control the gas quantity (gas volume) control module 55 in order to transport quantities or partial quantities of breathing gas mixture to the gas outlet 52.

In addition, the control and evaluation unit 70, 70′, 70″ is able to integrate events or situations of the flight operation 707 or special conditions 700 into the control and coordination of the module 50 and/or the supply of quantities of breathing gas mixture to the gas collection device 81. Such situations include, for example, states, in which it may be assumed that a gas component other than oxygen, nitrogen, moisture or water vapor and carbon dioxide may be present in the breathing gas mixture, as well as events 707 from flight operations (take-off, landing, descent, acceleration, refueling in the air, looping).

FIG. 3 shows a sequence 900 with a coordination of an operation of a monitoring system 100 according to FIG. 1 or FIG. 2 and a monitoring system 100 and a gas collection device 81 according to FIG. 2, beginning with a START 901.

The execution of the start 901 of the coordination with the configuration of further actions 902, 903, 905 can optionally take place in dependence 906 on a status signal 700 or in dependence 907 on situations of the flight operation 707—as explained in more detail in FIG. 2.

This is followed by an activation 902 of the module 50 for gas transportation and an activation 903 of the gas quantity control module 55 for a supply of defined quantities of breathing gas mixture from the monitoring system 100 (FIG. 2) to the gas collection device 81 (FIG. 2) and there by means of the gas distribution system 85 into the respective collection containers 82, 83, 84.

The activations 903, 902 can, for example, be time-controlled, as schematically indicated for activation 903, i.e. the supply of quantities of breathing gas mixture into the collection containers 82, 83, 84 takes place for a predetermined period of time, which is calculated such that sufficient filling of the collection containers 82, 83, 84 is ensured.

As soon as the supply of quantities of breathing gas mixture is completed, a deactivation 905 of the gas quantity control module 55 causes an end to the supply of quantities of breathing gas mixture to the gas collection device 81.

This is followed by a termination (STOP) 909 of the coordination of the operation of monitoring system 100 and gas collection device 81.

The gas transportation module 50 may also optionally remain deactivated 905 or optionally remain activated 902 at the end 909 of the sequence 900 in order to continue to deliver quantities of breathing gas mixture from the pilot 99 (FIG. 1) to the sensor system 60 (FIG. 1, FIG. 2).

In the sequence 900, an output signal can optionally be provided in a further step 907, which indicates an operating state 908—as explained in more detail in relation to FIG. 2—of the gas collection device 81.

While specific embodiments of the invention have been shown and described in detail to illustrate the application of the principles of the invention, it will be understood that the invention may be embodied otherwise without departing from such principles.

### REFERENCE SIGN LIST

10 Sample gas line for breathing gas mixture
20 Breathing mask
21 Gas connection to breathing mask
23 Connecting element
24, 25 Hose lines (tubes)
40 Operating and input elements
44 Display elements
46 Interface
50 Gas transportation module, pump P_{M}, gas extraction (pumping)
51 Gas inlet
52 Gas outlet
53 Admission
55 Gas quantity (gas volume) control module
60 Sensors
66 Gas sensor
70 Control and evaluation unit
70′, 70″ Control module
80 Collection gas line for collecting quantities of breathing gas mixture
81 Gas collection device
82, 83, 84 Collection containers, collection tubes, adsorption tubes
85 Gas distribution system
99 Person, pilot, aircraft pilot
100 Monitoring system
700 Special condition
707 Events or situations in flight operations
900-909 Coordination procedure

What is claimed is:

1. A monitoring system for monitoring a composition of a breathing gas mixture of a breathing gas supply of an aircraft pilot in aircraft or flying devices, the system comprising:
a sensor system comprising one or more gas sensors;
a gas collection device;
a gas quantity control module;
a gas transportation module configured to supply defined quantities of breathing gas mixture from a measuring location, via a gas sample line, to the monitoring system and to the sensor system and to the gas quantity control module; and
a control and evaluation unit operatively connected to the sensor system, the gas transportation module and the gas quantity control module,
wherein the control and evaluation unit is configured to control the sensor system, control the gas transportation module and control the gas quantity control module,
wherein the sensor system and the control and evaluation unit are configured to metrologically detect gases or gas mixtures,
wherein the control unit is configured to control a sequence of metrological monitoring of a gas composition of the breathing gas mixture in the aircraft or flying devices and to organize, control, manage or regulate the sequence,
wherein the gas quantity control module is configured to supply defined quantities of breathing gas mixture from the monitoring system to the gas collection device, and
wherein the collection device is configured to collect and store quantities of the breathing gas mixture.

2. A monitoring system according to claim 1, wherein the control and evaluation unit is configured as a plurality of control modules, wherein at least one control module is configured as a component of the monitoring system and wherein at least one further control module is configured as a component of the gas collection device.

3. A monitoring system according to claim 2, wherein the monitoring system and/or the gas collection device comprises at least one interface.

4. A monitoring system according to claim 3, wherein components of the monitoring system and the gas collection device are connected to one another by means of the at least one interface and the control and evaluation unit and/or the control modules are configured to carry out coordination of an operation of the gas collection device and the monitoring system by means of the interfaces.

5. A monitoring system according to claim 4, wherein the control and evaluation unit and/or the control modules are configured to receive a status signal at the at least one interface and to take the status signal into account when carrying out the coordination.

6. A monitoring system according to claim 4, wherein the control and evaluation unit and/or the control modules are configured, when carrying out the coordination to perform at least one action from the following group of actions:
activate a switching device or a changeover device for starting the supply of quantities of breathing gas mixture to the gas collection device;
deactivate a switching device or a changeover device for terminating the supply of quantities of breathing gas mixture to the gas collection device;
activate the gas transportation module;
activate the gas quantity control module and/or further components of the gas collection device for supplying and collection quantities of breathing gases in the gas collection device.

7. A monitoring system according to claim 1, wherein the gas collection device comprises at least one collection container.

8. A monitoring system according to claim 2, wherein the gas collection device comprises:
the further control module;
collection containers; and
a switching device for distributing the gas quantities to the collection containers.

9. A monitoring system according to claim 8, wherein the control and evaluation unit and/or the control modules coordinate the activation and/or deactivation of switching device as a function of measured values determined with the monitoring system and/or states of the breathing gas supply identified with the monitoring system.

10. A monitoring system according to claim 8, further comprising an additional sensor system configured to determine events and/or situations of flight operation, wherein the control and evaluation unit or one of the control modules coordinate the activation and/or deactivation of the switching device as a function of the determined events and/or situations of the flight operation.

11. A monitoring system according to claim 1, further comprising an output unit configured to enable an output signal to be provided which indicates an operating state of the gas collection device.

12. A process of operating a monitoring system and a gas collection device, the process comprising the steps of:
providing a monitoring system for monitoring a composition of a breathing gas mixture of a breathing gas supply of an aircraft pilot in aircraft or flying devices, the system comprising: a sensor system comprising one or more gas sensors; a gas collection device; a gas quantity control module; a gas transportation module configured to supply defined quantities of breathing gas mixture from a measuring location, via a gas sample line, to the monitoring system and to the sensor system and to the gas quantity control module; and a control and evaluation unit operatively connected to the sensor system, the gas transportation module and the gas quantity control module, wherein the control and evaluation unit is configured to control the sensor system, control the gas transportation module and control the gas quantity control module, wherein the sensor system and the control and evaluation unit are configured to metrologically detect gases or gas mixtures, wherein the control unit is configured to control a sequence of metrological monitoring of a gas composition of a breathing gas mixture in aircraft or flying devices and to organize, control, manage or regulate the sequence; wherein the gas quantity control module configured to supply defined quantities of breathing gas mixture from the monitoring system to the gas collection device; and wherein the collection device is configured to collect and store quantities of breathing gas mixture; and
coordinating an operation of the monitoring system and the gas collection device with a coordination sequence comprising:
starting the coordination of the operation of the monitoring system and gas collection device;
activating the gas transportation module and activating the gas quantity control module for a supply of defined quantities of breathing gas mixture from the monitoring system to the gas collection device;
deactivating the gas transportation module and/or deactivating the gas quantity control module to terminate the supply of quantities of breathing gas mixture to the gas collection device;
terminating the coordination of the operation of the monitoring system and the gas collection device.

13. A process according to claim 12, wherein the start of the sequence of steps is initiated in dependence on a provided status signal which indicates a status of the breathing gas supply or indicates events or situations of flight operation.

14. A process according to claim 12, further comprising the step of distributing and/or dividing quantities of breathing gas mixture into different gas collection containers.

15. A process according to claim 12, further comprising the step of providing an output signal is provided which indicates an operating state of the gas collection device.

16. A system comprising:
a monitoring system for monitoring a composition of a breathing gas mixture of a breathing gas supply of an aircraft pilot in aircraft or flying devices, the monitoring system comprising:
a sensor system comprising one or more gas sensors;
a gas quantity control module;
a gas transportation module configured to supply defined quantities of breathing gas mixture from a measuring location, via a gas sample line, to the monitoring system and to the sensor system and to the gas quantity control module; and
a control and evaluation unit operatively connected to the sensor system, the gas transportation module and the gas quantity control module, wherein the control and evaluation unit is configured to control the sensor system, control the gas transportation module and control the gas quantity control module, wherein the sensor system and the control and evaluation unit are configured to metrologically detect gases or gas mixtures, wherein the control unit is configured to control a sequence of metrological monitoring of a gas composition of a breathing gas mixture in aircraft or flying devices and to organize, control, manage or regulate the sequence; and
a gas collection device operatively connected to the monitoring system and configured to collect and store quantities of the breathing gas mixture, wherein the gas quantity control module is configured to supply defined quantities of breathing gas mixture from the monitoring system to the gas collection device.

17. A system according to claim 16, wherein the control and evaluation unit is configured as a plurality of control modules, wherein at least one control module is configured as a component of the monitoring system and wherein at least one further control module is configured as a component of the gas collection device.

18. A system according to claim 17, further comprising an interface connecting components of the monitoring system and the gas collection device to one another by means of the at least one interface, wherein the control and evaluation unit and/or the control modules are configured to carry out coordination of an operation of the gas collection device and an operation of the monitoring system via the interface.

19. A system according to claim 18, wherein the control and evaluation unit and/or the control modules are configured, when carrying out the coordination to perform at least one action from the following group of actions:
activate a switching device or a changeover device for starting the supply of quantities of breathing gas mixture to the gas collection device;
deactivate a switching device or a changeover device for terminating the supply of quantities of breathing gas mixture to the gas collection device;
activate the gas transportation module;
activate the gas quantity control module and/or further components of the gas collection device for supplying and collection quantities of breathing gases in the gas collection device.

20. A system according to claim 17, wherein the gas collection device comprises:
the further control module;
collection containers; and
a switching device for distributing the gas quantities to the collection containers.
